(19) United States
(12) Patent Application Publication
(10) Pub. No.: US 2004/0224935 A1
(43) Pub. Date: Nov. 11, 2004

(54) Topical antiandrogenic steroids

(75) Inventors: Fernand Labrie, Quebec (CA); Louis Provencher, Charney (CA); Sylvain Gauthier, Quebec (CA)

Correspondence Address: OSTROLENK FABER GERB & SOFFEN, 1180 AVENUE OF THE AMERICAS, NEW YORK, NY, 100368403

(73) Assignee: Endorecherche, Inc.

(21) Appl. No.: 10/815,515

(22) Filed: Mar. 31, 2004

Related U.S. Application Data
(60) Provisional application No. 60/461,069, filed on Apr. 7, 2003.

(51) Int. Cl. A61K 31/56; C07J 1/00

(52) U.S. Cl. 514/182; 552/625

(57) ABSTRACT

Steroidal antiandrogens and pharmaceutical compositions thereof, are used for reduction of the risk of developing, or for treatment of, androgen-dependent skin related diseases. In preferred embodiments, the antiandrogen EM-3180 is used for reduction of the risk of developing, or the treatment, of acne, seborrhea, hirsutism or androgenic alopecia:

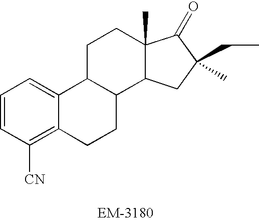

Methods of treatment utilize the antiandrogen alone, or in combination with other active ingredients such as an inhibitor of a 5α-reductase, an inhibitor of type 5 17β-hydroxysteroid dehydrogenase, and/or an inhibitor of prostate short-chain dehydrogenase/reductase.

### BACKGROUND OF THE INVENTION

This invention relates to novel inhibitors of sex steroid activity, such as antiandrogenic compounds that have effective antagonistic activity while substantially lacking agonistic effects. More particularly, the invention relates to certain steroid derivatives which block androgen action by acting, among other mechanisms, through the androgen receptors but not activating such receptors. These compounds are useful in the treatment of (or reduction of risk of acquiring) androgen-exacerbated skin diseases, discussed herein.

### BRIEF DESCRIPTION OF THE PRIOR ART

During the treatment of certain androgen dependent diseases, it is important to greatly reduce or, if possible, to eliminate androgen-induced effects. For this purpose, it is desirable to both block access to the androgen receptors with “antiandrogens”, thus preventing androgens from binding and activating those receptors, and also to reduce the concentration of androgens available to activate the receptors. It is possible that, even in the absence of androgens, unoccupied androgen receptors may be biologically active. Hence, antiandrogens which bind and block the receptors may produce better therapeutic results than therapy which only inhibits androgen production.

Antiandrogens may have a significant therapeutic effect in slowing or stopping the progress of androgen-dependent diseases, e.g. diseases whose onset or progress is aided by androgen receptor or androgen receptor modulator activation.

It is desired that an antiandrogen used in therapy to reduce androgen receptor activation have both good affinity for the androgen receptor and a substantial lack of inherent androgenic activity. The former refers to the ability of an antiandrogen to bind to the androgen receptor, and thus to block access to the receptor by androgens. The latter refers to the effect that the antiandrogen has on the receptor once it binds thereto. Some antiandrogens may possess inherent androgenic activity (“agonistic activity”) which undesirably activates the very androgen receptors whose activation they are intended to prevent In other words, an antiandrogen with intrinsic androgenic activity may successfully bind to androgen receptors, desirably blocking access to those receptors by natural androgens, yet may undesirably itself activate the receptor.

Known non-steroidal antiandrogens such as flutamide, casodex and anandron lack undesirable androgenic activity, but may not have receptor affinity as good as steroidal antiandrogens (i.e. androgen derivatives having a steroidal nucleus that is modified to provide antiandrogenic activity). Steroidal antiandrogens, however, are believed more likely to possess undesirable agonistic characteristics.

Most of known antiandrogens such as flutamide have unwanted systemic activity when applied on the skin. For androgen-dependent skin related diseases such as acne, hirsutism, seborrhea, androgenic alopecia and premature male baldness, it is preferred that antiandrogens not penetrate in the body in significant amount and have antiandrogenic effect in other tissues than on the area of the skin where they are applied.

Thus, there is a need in the Art for steroidal antiandrogens having good affinity to the androgen receptor and substantially lacking undesirable agonistic and systemic characteristics.

### SUMMARY OF THE INVENTION

It is an object of the present invention to provide steroidal antiandrogens, having good affinity for the androgen receptor, while substantially lacking androgenic and systemic activity. These antiandrogens may be useful in the treatrnent of androgen-dependent skin related diseases as described in more detail infra.

In one aspect, the invention provides an antiandrogenic compound of the molecular formula:

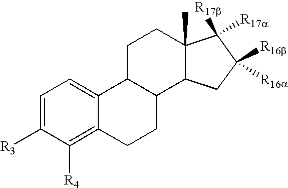

wherein R₃ is selected from the group consisting of hydrogen, fluoro, chloro, bromo, iodo and a moiety —C≡CR′(R′ being hydrogen or C1-C6 lower alkyl); wherein R₄ is selected from the group consisting of hydrogen, fluoro, chloro, bromo, iodo, and cyanide:

wherein R_{17α}, is selected from the group consisting of hydrogen, C1-C6 lower alkyl C2-C6 lower alkenyl, and C2-C6 lower alkynyl, or R_{17α}, and R_{17β} together are oxygen forming a keto group;

wherein R_{17β} is selected from the group consisting of hydroxyl and a group transformed on the skin into hydroxyl, or R_{17α} and R_{17β} together are oxygen forming a keto group;

wherein R_{16α} is selected from the group consisting of hydrogen, C1-C6 lower alkyl, C2-C6 lower alkenyl, and C2-C6 lower alkynyl;

wherein R_{16β} is selected from the group consisting of hydrogen, C1-C6 lower alkyl, C2-C6 lower alkenyl, and C2-C6 lower alkynyl;

wherein at least one of R₃ or R₄ is not an hydrogen.

In another aspect, the invention provides topical pharmaceutical compositions containing the antiandrogens of the invention together with pharmaceutically acceptable diluents or carriers.

In another aspect, the novel antiandrogens, or pharmaceutical compositions containing them, are used in the treatment or prevention of androgen-dependent skin related diseases such as acne, hirsutism, seborrhea, androgenic alopecia, premature male baldness and the like.

It is another object to provide treatment and prevention regimens for androgen sensitive skin related diseases which regimens include use of androgen receptor antagonists disclosed herein.

### DETAILED DESCRIPTION OF THE PREFERRED EMBODIMENTS

The antiandrogens and pharmaceutical compositions containing them, may be utilized in accordance with the invention to treat or reduce the risk of acquiring androgen-sensitive skin-related diseases whose progress or onset is aided by activation of androgen receptors. These include but are not limited to acne, seborrhea, hirsutism, androgenic alopecia, premature male baldness, and the like.

It is preferred that the R₄ substituent be a cyanide group

The following compounds, EM-3221, (4cyano-16α-methyl-16β-ethyl-1,3,5(10)-estratrien-17β-ol; 16-Ethyl-17-hydroxy-13,16-dimethyl-7,8,9,11,12,13,14,15,16,17-decahydro-6H-cyclopent[α]phenanthrene4-carbonitrile) and EM-3180 (4-cyano-16α-methyl-16β-ethyl-1,3,5(10)-estratrien-17-one; 16-Ethyl-17-oxo-13,16-dimethyl-7,8,9,11,12,13,14,15,16,17-decahydro-6H-cyclopent[α]phenanthrene-4-carbonitrile) are especially preferred:

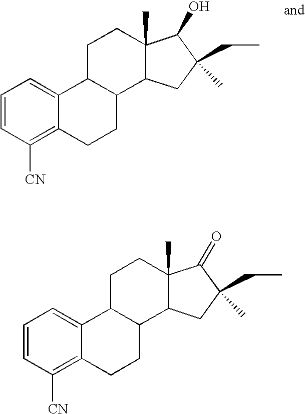

The antiandrogens of the invention are preferably formulated together with pharmaceutically acceptable diluent, excipient or carrier into pharmaceutical compositions at conventional antiandrogen concentrations for antiandrogens used in the prior art The attending clinician may elect to modify the concentration and/or dosage in order to adjust the dose to the particular response of each patient. Preferably, the attending clinician will, especially at the beginning of treatment, monitor an individual patient's overall response to treatment, adjusting dosages as necessary where a given patients' reaction to treatment is atypical. As discussed in more detail below, carriers, excipients or diluents include liquids. When a composition is prepared other than for immediate use, an art-recognized preservative is typically included (e.g. benzyl alcohol). The novel pharmaceutical compositions of the invention may be used in the treatment of androgen-related skin diseases, or to reduce the likelihood of acquiring such diseases.

The antiandrogens of the invention are utilized for the treatment of androgen related diseases of the skin such as acne, seborrhea, hirsutism, androgenic alopecia and premature male baldness. The antiandrogens are preferably administered topically together with a conventional topical carrier or diluent. It is preferred that the diluent or carrier does not promote transdernal penetration of the active ingredients into the blood stream or other tissues where they might cause unwanted systemic effects.

When the compound is administered in a cutaneous or topical carrier or diluent, the carrier or diluent may be chosen from any known in the cosmetic and medical arts, e.g. any gel cream, lotion, ointment, liquid or non liquid carrier, emulsifier, solvent, liquid diluent or other similar vehicle which does not exert deleterious effect on the skin or other living animal tissue. The carrier or diluent is usually a mixture of several ingredients, including, but not limited to liquid alcohols, liquid glycols, liquid polyalkylene glycols, water, liquid amides, liquid esters, liquid lanolin, lanolin derivatives and similar materials. Alcohols include mono and polyhydric alcohols, including ethanol, glycerol, sorbitol, isopropanol, diethylene glycol, propylene glycol, ethylene glycol, hexylene glycol, mannitol and methoxyethanol. Typical carriers may also include ethers, e.g. diethyl and dipropyl ether, methoxypolyoxyethylenes, carbowaxes, polyethyleneglycerols, polyoxyethylenes and sorbitols. Usually, the topical carrier includes both water and alcohol in order to maximize the hydrophylic and lipophylic solubility, e.g. a mixture of ethanol or isopropanol with water.

A topical carrier may also include various other ingredients commonly used in ointments and lotions and well known in the cosmetic and medical arts. For example, fragrances, antioxidants, perfumes, gelling agents, thickening agents such as carboxymethylcellulose, surfactants, stabilizers, emollients, coloring agents and other similar agents may be present.

The concentration of active ingredient in the ointment, cream, gel or lotion is typically from about 0.01 to 20 percent, preferably between 0.1 and 10 percent, and most preferably 1 percent (by weight relative to the total weight of the lotion, cream, gel or ointment). Within the preferred ranges, higher concentrations allow a suitable dosage to be achieved while applying the lotion, ointment, gel or cream in a lesser amount or with less frequency.

Several non-limiting examples infra describe the preparation of a typical lotion and gel, respectively. In addition to the vehide used in those examples, one skilled in the art may choose other vehides in order to adapt to specific dermatologic needs.

For topical use lotion, ointment, gel or cream should be thoroughly rubbed into the skin so that no excess is plainly visible, and the skin is preferably not washed in that region for at least 30 minutes. The amount applied should provide at least 0.001 milligrams of antiandrogen per square centimeter (preferably from 0.01 to 1 mg/cm2) per application. It is desirable to apply the topical composition to the effected region from 1 to 6 times daily, e.g. 3 times daily at approximately regular intervals.

Prostate Short-Chain Dehydrogenase Reductase 1 (PSDR1) was first identified as a Short-Chain Steroid Dehydrogenase/Reductase that is highly expressed in Normal and Neoplastic Epithelium (Lin et aL, Cancer Research 61:1611-8, 2001) without enzymatic activity characterization. Recently, using a protein overexpressed in SF9 insect cells, the enzyme has been found to have retinal reductase activities catalyzing the transformation of retinal into retinol (Kedishvili et al., JBC 277, 28909-15, 2002). The authors affirmed that the enzyme is selective for retinoids and does not possess any significant oxidative or reductive activity toward the functional hydroxyl or keto groups in positions 3, 17, or 20 of steroids.

In contrast, using human embryonic kidney cells stably transfected with human PSDR1 cDNA in culture, we have found that the enzyme possesses a predominant 17β-hydroxysteroid dehydrogenase activity, selective for 5α-reduced steroids, catalyzing the transformation of 5α-androstane-3,17-dione (5α-dione) into 5α-androstane-17β-ol-3-one (dihydrotestosterone, DHT) and of 5α-androstane-3α-ol-17-one (ADT) into 5α-androstane-3α,17β-diol (3α-diol).

Using RealTime PCR to quantify the mRNA expression levels of the enzyme in various human and mouse tissues, we showed that this enzyme is widely expressed. It is strongly expressed in the human prostate, and at a lesser extend in the human liver, adrenal and placenta. In the mouse, it is highly expressed in the testis and in the preputial and clitoral glands. It is also expressed in mouse seminal vesicles, epididymis, hypophysis, adrenals, liver, kidney, thymus, adipose tissue, skin, lung, esophagus, colon, mammary gland, uterus, vagina, and ovary.

These results strongly suggest that this enzyme plays an important role in the formation of the most potent natural androgen DHT.

In some embodiments of the invention, the antiandrogen of the invention is used in combination with another active ingredient as part of a combination therapy. For example, the novel antiandrogen may be utilized together with a separate 5α-reductase inhibitor, a type 5 17β-hydroxy steroid dehydrogenase inhibitor, or Prostate Short-Chain Dehydrogenase Reductase 1 inhibitor which may be incorporated into the same pharmaceutical composition as is the antiandrogen, or which may be separately administered. Combination therapy could also include treatment with one or more compounds which inhibit the production of dihydrotestosterone or its precursors. In some preferred embodiments of the invention, the topical pharmaceutical composition further includes an inhibitor of steroid 5α reductase activity. One such inhibitor (“Propecia”) is commercially available form Merck Sharp and Dohme. Inhibitors of type 5 17β-hydroxysteroid dehydrogenase, more particularly compound EM-1404, are disdosed in the international publication WO 99/46279

EM-1791, one inhibitor of Prostate Short-Chain Dehydrogenase Reductase-1, can be easily synthesized from Benzypyran compounds disdosed in U.S. Pat. No. 6,060,503 as described in the following scheme:

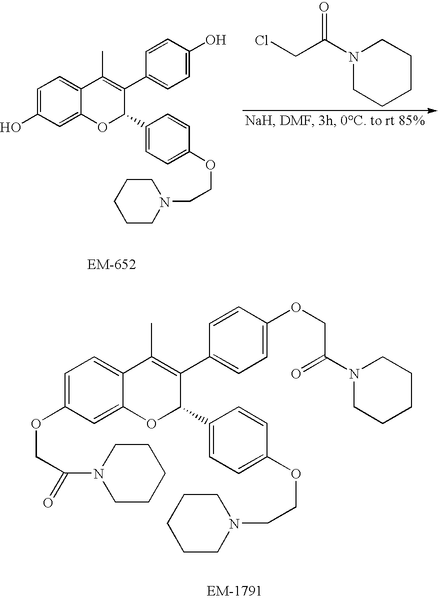

A patient in need of treatment or reducing the risk of onset of a given disease is one who has either been diagnosed with such disease or one who is susceptible to acquiring such disease. The invention is especially useful for individuals who, due to heredity, environmental factors or other recognized risk factor, are at higher risk than the general population of acquiring the conditions to which the present invention relates.

Except where otherwise stated, the preferred dosage of the active compounds of the invention is identical for both therapeutic and prophylactic purposes. The dosage for each active component discussed herein is the same regardless of the disease being treated (or prevented).

Where two are more different active agents are discussed as part of a combination therapy herein (e.g. an enzyme inhibitor and an antiandrogen), a plurality of different compounds are administered rather than a single compound having multiple activities.

Except where otherwise indicated, the term “compound” and any associated molecular structur may indude any possible stereoisomers thereof, in the form of a racemic mixture or in optically active form.

Except where otherwise noted or where apparent from context, dosages herein refer to weight of active compounds unaffected by pharmaceutical excipients, diluents, carries or other ingredients, although such additional ingredients are desirably included, as shown in the examples herein. Any dosage form (cream, gel ointment or the like) commonly used in the pharmaceutical industry is appropriate for use herein, and the terms “excipient”, “diluent” or “carrier” include such non-active ingredients as are typically included, together with active ingredients in such dosage forms in the industry.

All of the active ingredients used in any of the combination therapies discussed herein may be formulated in pharmaceutical compositions which also include one or more of the other active ingredients. Alternatively, they may each be administered separately or otherwise enjoys the benefits of each of the active ingredients (or strategies) simultaneously. In some preferred embodiments of the invention, for example, one or more active ingredients are to be formulated in a single pharmaceutical composition. In other embodiments of the invention, a kit is provided which includes at least two separate containers wherein the contents of at least one container differs, in whole or in part, from the contents of at least one other container with respect to active ingredients contained therein. Two or more different containers are used in the combination therapies of the invention. Combination therapies discussed herein also include use of one active ingredient of the combination in the manufacture of a medicament for the treatment (or prevention) of the disease in question where the treatment or prevention further includes another active ingredient or strategy of the combination.

When 5α-reductase inhibitors are used in combination therapies, in accordance with the invention described herein, oral or cutaneous dosage is preferably between 0.1 mg and 100 mg per day per 50 kg body weight, more preferably between 0.5 mg/day and 10 mg/day, for example 1 mg per day of finasteride.

When type 5 17β-hydroxysteroid dehydrogenase inhibitors are used in combination therapies, in accordance with the invention described herein, oral or cutaneous dosage is preferably between 5 mg and 500 mg per day per 50 kg body weight, more preferably between 10 mg/day and 400 mg/day, for example 300 mg per day of EM-1404.

When PSDR-1 inhibitors are used in combination therapies, in accordance with the invetion described herein, cutaneous dosage is preferably between 10 mg and 2000 mg per day per 50 kg body weight, more preferably between 100 mg/day and 1000 mg/day, for example 500 mg per day of EM-1791.

### PREFERRED ANTIANDROGENS

Set forth in the table below is a list of compounds which we have found to be useful as antiandrogens for topical application (Hamster's ear sebaceous gland assay). The table also includes in vitro determination of androgenic/antiandrogenic activity on mouse mammary carcinoma Shionogi cells and in vivo determination of systemic antiandrogenic activity in immature male rats. It is believed that the rat assays are better for predicting systemic efficacy, while the Shionogi assays are better for predicting efficacy against skin diseases.

TABLE 1
TOPICAL ANTIANDROGENS

| | | | | RAT | | | |
|---|---|---|---|---|---|---|---|
| | | | | (in vivo) | | | |
| | | | | (systemic) | | | |
| | | Antiandrogenic activity | | Prostate | SV | | |
| | | in Hamster's | | Inh. | Inh | Shionogis | |
| | | sebaceous glands | | % inh. | % inh. | (In Vitro) | |
| | | | Inh. Vs | (0.5 mg, | (0.5 mg, | | VS |
| NAME | STRUCTURE | Dose | Cx (%) | per os) | per os) | IC₅₀(nM) | OH-FLU* |
| 1 | 2 | 3 | 4 | 5 | 6 | 7 | 8 |
| EM-3180 | 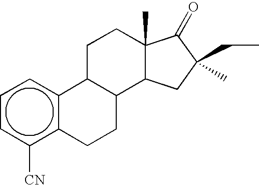 | 1 μg: 3 μg: 10 μg; | 57.1 90.5 95.2 | 0 | 0 | 35 | 2 |
| EM-3497 | 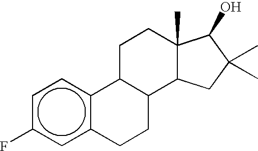 | 1 μg: 3 μg: 10 μg: | 62.9 62.9 77.7 | 0 | 0 | 46 | 1.5 |
| EM-3166 | 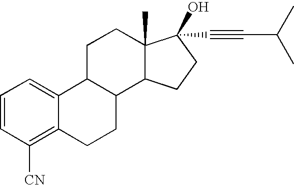 | 1 μg: 3 μg: 10 μg: | 47.6 66.6 76.2 | 0 | 15 | 21.8 | 5.0 |
| EM-3144 | 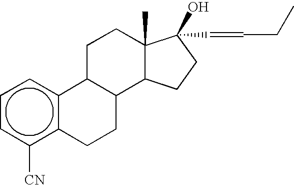 | 1 μg: 3 μg: 10 μg: | 9.5 71.4 71.4 | 8 | 12 | 4.4 | 8.5 |
| EM-3852 | 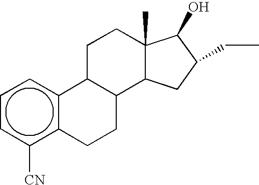 | 1 μg: 3 μg: 10 μg: | 51.8; 28.6 66.6; 66.6 70.4; 85.7 | 0 | 0 | 16.1 | 8.7 |
| EM-3411 | 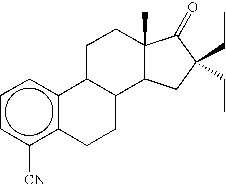 | 1 μg: 3 μg: 10 μg: | 3.7 14.8 62.9 | 0 | 10 | | ˜1 |
| EM-3223 | 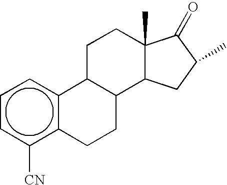 | 1 μg: 3 μg: 10 μg: | 25.9 37.0 59.2 | 0 | 18 | 1.3 6.5 | 16 14 |
| EM-3217 | 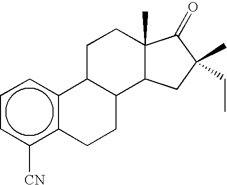 | 1 μg: 3 μg: 10 μg: | 4.8 0 42.8 | 0 19 | 19 0 | | ˜1 |
| EM-3178 | 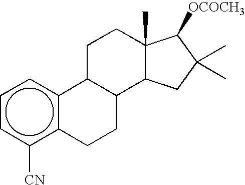 | 1 μg: 3 μg: 10 μg: | 9.5 9.5 38.1 | 0 | 0 | 17 | 4.1 |
| EM-3221 | 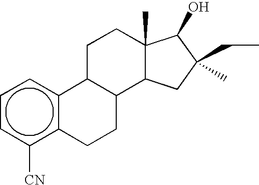 | 1 μg: 3 μg: 10 μg: | 0 9.5 33.3 | 0 | 1 | 3.2 | 6.6 |
| EM-3226 | 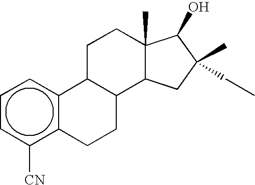 | 1 μg: 3 μg: 10 μg: | 7.4 29.6 33.3 | 0 | 0 | 3.6 | 5.9 |
| EM-3415 | 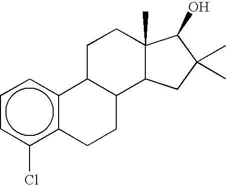 | 1 μg: 3 μg: 10 μg: | 0 11.1 44.4 | 0 | 0 | 26 | 2.5 |
| EM-3473 | 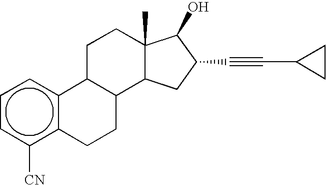 | 1 μg: 3 μg: 10 μg: | 7.4 0 14.8 | 0 | 0 | 9.3 | 9 |
| EM-4142 | 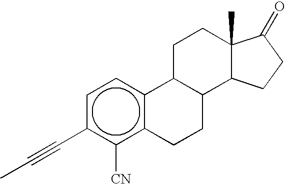 | 1 μg: 3 μg: 10 μg: | 18.5 18.5 37.0 | 0 | 22 | 29 | 3.7 |
| EM-5035 | 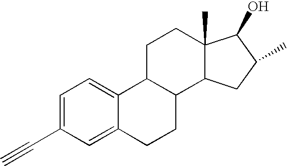 | 1 μg: 3 μg: 10 μg: | 3.7 11.1 51.9 | 11 | 4 | 66 | 1.6 |
| EM-3165 | 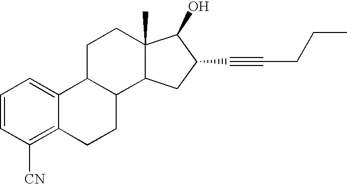 | 1 μg: 3 μg: 10 μg: | 19.0 14.3 23.8 | 1 | 14 | 15.1 | 7.2 |
| EM-3803 | 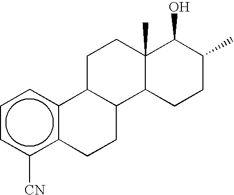 | 1 μg: 3 μg: 10 μg: | 33.3 51.8 37.0 | 0 | 30 | 11 8.6 | 10.6 10.3 |
| EM-4157 | 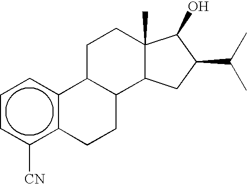 | 1 μg: 3 μg: 10 μg: | 18.5 29.6 40.7 | 0 | 55 | 33 | 5 |
| EM-2557 | 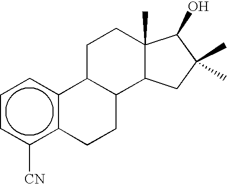 | 1 μg: 3 μg: 10 μg: | 52.4 42.8 66.6 | 6 | 0 | 15 18 | 7.2 6.8 |
| EM-2627 | 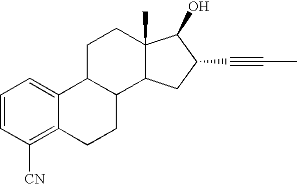 | 1 μg: 3 μg: 10 μg: | 14.3 42.8 66.6 | 0 | 25 | 7.8 | 8.3 |

Cx: CASTRATED
* OH-Flu = 1

Legend:

Column 3 represents the dose of antiandrogenic compounds applied on the left ear of the intact male hamster.

Column 4 represents the relative inhibition in % of the area of the sebaceous gland of the left ear versus control hamster. Higher values approaching the effect of castration are preferable

Column 5 represents the % of antiandrogenic efficacy in rat prostate, relatively to the percentage of inhibition of flutamide calculated by the formula:

% efficacy versus Flu=100×% inhib (compound)/% inhib (Flu).

Where the percentage of inhibition (% inhib) is calculated by the following formula:

% lnhib=100−[W (compound)−W (control)/W (DHT)−W (control)]×100.

W is the weight of the prostate.

Higher values are preferable.

Column 6 represents the % of antiandrogenic efficacy on the rat seminal vesicle, relative to the percentage of inhibition by flutamide calculated by the formula:

% efficacy versus Flu=100×% inhib (compound)/% inhib (Flu).

Where the percentage of inhibition (% inhib) is calculated by the following formula:

% Inhib=100−[W (compound)−W (control)/W (DHT)−W (control)]×100.

W is the weight of the seminal vesicle.

Higher values are preferable.

Column 7 represents the dose (expressed in nM) that inhibits by 50% (IC₅₀) DHT-stimulated Shionogi mouse mammary carcinoma cell number. Lower values are preferable.

Column 8 represents the ratio of Inhibition Constant (Ki) value of the inhibition of DHT-stimulated Shionogi mouse mammary carcinoma cell number by hydroxyflutamide versus the effect of the tested-compound. Higher values are preferable.

### EFFICACY OF THE PREFERRED INHIBITORS

In vivo Assays of Topical Antiandrogenic Activity

The Antiandrogenic activity of antiandrogenic compounds has been measured using a Histomorphometry assay of the Ear Sebaceous Glands in the Hamster

1. Animals Male Golden Syrian, Hamster (SYR) of 110-120 g were obtained from Harlan Sprague Dawlay (Madison, USA) and housed up to 2 per cage in plastic cages in a temperature (22±3° C.) and light (12 h light/day, lights on at 7 h 15) in a controlled environment. The hamsters were fed with Certified Rodent Diet 5002 (pellet) and tap water ad libitum. The animals were acclimated for at least five days before the beginning of the study. Animals were randomly assigned to groups of eight hamsters. One group of hamsters was castrated under isoflurane-induced anesthesia on the day of dosing initiation (SD 1) and used as control group.

2. Treatments To evaluate the antiandrogenic activity, tested compounds were applied topically on the inner part of the left ear, once daily, for 14 days. Ten μL solution of acetone: ethanol: propylene Glycol (1:1:2; v:v:v) containing 0.1, 0.3 or 1.0 mg/mL of tested compound was carefully applied onto a region between the two cartilage ridges of the ventral surface of left pinna. For animals in the castrated and intact control groups ten-μL vehicle was applied onto the left ear. No solution will be applied on the right ear for any animals of the study.

3. Postmortem Observations and Measurements On Study Day 15, the hamsters were euthanized by cervical dislocation under isoflurane anesthesia. The left and right ears were collected attached together by the head skin, flat fixed on a paper and were immersed in 10% neutral buffered formalin. From the flat fixed ear and using paper puncture that makes a hole of 6 mm, the region where solution was applied was collected from each ear. Using a scalpel blade, the collected 6 mm round ear specimen was cut in the middle between the two cartilage ridges. The two equal parts of the ear round specimen were embedded in paraffin. After processing the tissue, the two parts were vertically embedded parallel to each other in a way that the flat 6 mm area was facing out. From each paraffin block, one section (5 μm thick) was cut and collected on a glass slide. Thus, each slide contained two elongated sections of 6 mm length. Slides were stained with hematoxylin and eosin.

4. Analysis of Sebaceous Gland Area Using the video camera and objective lens number X5 of the light microscope, the resulting field that appears on the screen has a length of 0.953 mm. From the glass slide, when we have examined the first 6 mm long section from the left to the right, the first and second fields were ignored and the third and fourth fields were captured for analysis by the image analyzer. Each field has the length of 0.953 mm. With the help of the screen mouse, the sebaceous glands within the whole field length (0.953mm) were marked. Also, an area with the length of the whole field and the height between stratum granulosum and the upper edge of the cartilage was drawn.

The image analyzer calculated the total area of the sebaceous glands (μm²) in each examined field. We also obtained the area, which has the length of 0.953 mm and the height between stratum granulosum and the cartilage. In addition, the percentage of the area occupied by the glands was obtained. Thus, for each ear, two sections were cut and two fields from each section were analyzed. The total of the four readings was averaged and the mean standard of error calculated by the image analyzer. The results were expressed in μm² as the total surface of glands per field and also as percentage of the area occupied by the glands.

B In vitro Assays of Androgenic/Antiandrogenic Activity of antiandrogenic compounds

The androgenic/antiandrogenic activity of preferred compounds has been measured using the Shionogi mouse mammary carcinoma cells.

1. Materials Minimal essential culture medium (MEM), non-essential amino acids, and fetal calf serum were purchased from Flow Laboratories. In order to remove endogenous steroids, serum was incubated overnight at 4° C. with 1% activated charcoal (Norit A, Fisher) and 0.1% Dextran T-70 (Pharmacia). A 2-h supplementary adsorption was performed at 25° C. in order to further remove protein-bound steroids. Serum was also inactivated by a 20-min incubation at 56° C.

5α-dihydrotestosterone (DET) was obtained from Steraloids. The antiandrogen hydroxyflutamide (OH-FLU) was kindly supplied by Drs. T. L. Nagabuschan and R. Neri (Schering Corporation, Kenilworth, U.S.A.).

2. Cell dispersion, culture and cloning Shionogi male mice bearing androgen-sensitive mammary tumors were obtained from Drs. Keishi Matsumoto, Osaka, Japan, and Yvonne Lefebvre, Ottawa, Canada. For primary culture, tumors were excised and washed in ice-cold sterile 25 mM Hepes buffer (137 mM NaCl; 5 mM KCl; 0.7 mM Na2HPO₄; 10 mM glucose, pH 7.2). After mincing with scissors, the tumor minces were digested for 2 h at 37° C. in Hepes buffer containing 3.8 mg/ml collagenase (Clostridium, Boehringer), 1.5 mg/ml hyaluronidase II (Sigma), and 3% bovine serum albumin fraction V (Schwartz-Mann). Dispersed cells were collected by centrifugation (500×g for 10 min), washed twice by suspension in minimal essential medium (MEM) containing 5% dextran-coated charcoal-treated fetal calf serum (DCC-FCS), 1% non-essential amino acids, 10 IU/ml penicillin, 50 μg/ml streptomycin, and 100 nM dihydrotestosterone (DHT) (Steraloids).

Cells were plated in the same medium at a density of 75 000 cells/ml in 75 cm² flasks under an atmosphere of 5% carbon dioxide in air at 37° C. The medium was changed weekly. Tested compounds were dissolved in ethanol and kept in stock solutions chosen to yield final ethanol concentrations less than 0.01% in the culture medium. Such a concentration of ethanol does not affect cell growth.

Cells were subcultured at near-confidence by gentle digestion in a solution of 0.1% pancreatin (Flow Laboratories) in Hepes buffer containing 3 mM ethylenediaminetetraacetic acid (EDTA) (pH 7.2). Cells were pelleted by centrifugation, resuspended in culture medium, counted in a Coulter counter, and replated as described above. Soft agar cloning was performed as described (Stanley et al., Cell 10: 35-44, 1977) in the presence of 100 nM DHT.

3. Measurement of Cell Growth Cells were plated in 24-well plates at a density of 20 000 cells/well. The indicated increasing concentrations of agents were added to triplicate dishes, and cells were grown for 10-12 days with changes of medium every 3-4 days. Cell number was measured by direct counting in a Coulter counter.

4. Calculations and Statistical Analysis IC₅₀ values of tested compounds were calculated according to a least-square regression as described by Rodbard (Endocrinology, 94, 1427-1437, 1974) Statistical significance was calculated according to Kramer multiple-range test.

C Systemic antiandrogenic activity (immature male rats)

1. Animals Immature male rats (Crl:CD(SD)Br) 22 to 24-day old were obtained from Charles-River, Inc. (St-Constant, Quebec, Canada) and housed up to 5 per cage in plastic bins in a temperature (23±1° C.)—and light (12 h light/day, lights on at 7h15)—controlled environment. The rats were fed rodent chow and tap water ad libitum. The day following their arrival, the animals were orchidectornized (CX) under Isoflurane anesthesia via scrotal route and randomly assigned to groups of 5 animals. One silastic implant of dihydrotestosterone (DHT; length of implant: 1 cm) was inserted subcutaneously in the dorsal area of animals at the time of orchidectomy. One group of 5 animals was CX only as control (no DHT implant inserted).

2. Treatments To evaluate the antiandrogenic activity, tested compounds were administered orally by gavage once daily at a dose of 0.5 mg/animal for 7 days (SD 1 to 7). Compounds were solubilized (when possible) in dimethylsulfoxide (DMS, 10% final concentration) and administered as suspension in 0.4% methylcellulose. Rats in CX control and CX+DHT control groups received the vehicle alone during the 7-day period. One group of animals received the antiandrogen Flutamide as reference. The animals were killed by cervical dislocation under isoflurane anesthesia on the 8th morning following castration. The ventral prostate and seminal vesicles were rapidly dissected and weighed.

3. Calculations and Statistical Analysis The percentage of inhibition (% inhib) is calculated by the following formula:

% Inhib=100−[W (compound)−W (control)/W (DHT)−W (control)]×100.

This percentage is reported as % of efficacy, relatively to the percentage of inhibition of flutaride calculated by the formula: % efficacy versus Flu=100×% inhib (compound)/% inhib (Flu).

W is the weight of the prostate or the seminal vesicle.

Some non-limiting examples of preferred active compounds are discussed below together with preferred synthesis techniques.

### EXAMPLES OF SYNTHESIS OF PREFERRED INHIBITORS

Proton NMR spectra were recorded on a Brucker AC-F 300 instrument or a Brucker Avance 400 MHz . The following abbreviations have been used: s, singlet; d, doublet; dd, doublet of doublet; t, triplet; q, quadruplet; and m, multiplet. The chemical shifts (δ) were referenced to chloroform (7.26 ppm for ¹H and 77.00 ppm for ¹³C) and were expressed in ppm. Thin-layer chromatography (TLC) was performed on 0.25 mm Kieselgel 60F254 plates (E. Merck, Darmstadt, FRG). For flash chromatography, Merck-Kieselgel 60 (230-400 mesh A.S.T.M.) was used. Unless otherwise noted, starting material and reactant were obtained commercially and were used as such or purified by standard means. All solvents and reactants purified and dried were stored under argon. Anhydrous reactions were performed under an inert atmosphere, the set-up assembled and cooled under argon. Organic solutions were dried over magnesium sulfate, evaporated on a rotatory evaporator and under reduced pressure. Starting materials and reagents were available from Sigma-Aldrich Canada Ltd. (Oakville, Ontario)

### LIST OF ABBREVIATIONS

BINAP 2,2′-Bis(diphenylphosphino)-1,1′binaphthyl

DMAP Dimethylaminopyridine

DMF Dimethyl formamide

Dppf 1,1′-bis(diphenylphosphino)ferrocene

HPLC High pressure liquid chromatography

KHMDS Potassium (hexamethyldisilyl)amide

LiHMDS Lithium (hexamethyldisilyl)amide

NBS N-bromosuccinimide

THF Tetrahydrofuran

Tf₂O Triflic anhydride

TMS Tetramethylsilyl

Pd₂(dba)₃ T ris dibenzylideneacetone dipalladium

### EXAMPLE 1

Synthesis of 3fluoro-16,16-dimethyl-1,3,5(10)-estratrien-17β-ol (EM-3497)

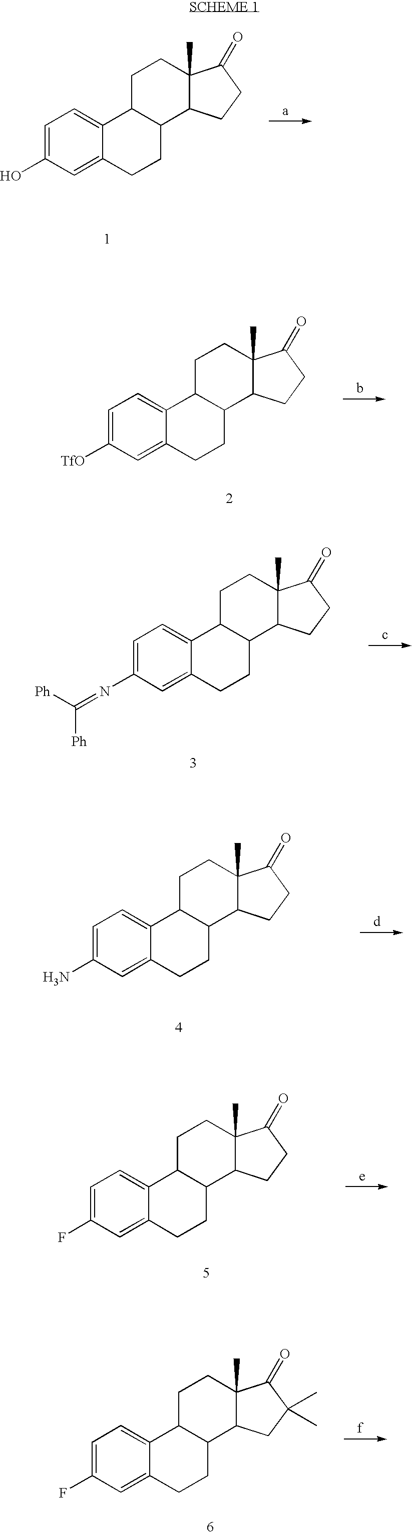

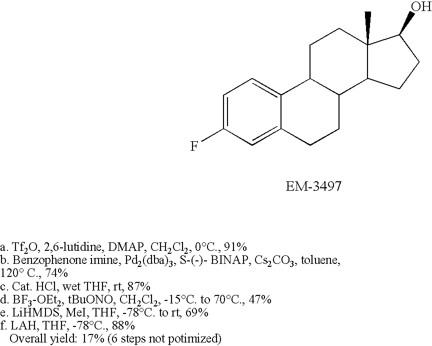

3-trifluoromethanesulfonate-1,3,5(10)-estratrien-17-one (2) Under argon atmosphere, a solution of compound 1 (500 mg, 1.95 nmuol), 2,6-lutidine (0.512 mL, 4.40 mmol) and 4-dimethylaminopyridine (48 mg, 0.39 mmol) in dichloromethane (25 mL) was cooled at 0° C., treated with trifluoromethanesulfonic anhydride (0.444 mL, 2.64 mmol) and stirred for 45 min. The reaction mixture was quenched with water and extracted with dichloromethane. The organic phase was washed with 2% hydrochloric acid, saturated sodium bicarbonate and water, dried over magnesium sulfate filtered, and evaporated. The crude oil was purified by flash chromatography (hexanes-ethyl acetate/49-1 to hexanes-ethyl acetate/4-1) to provide trifluoromethanesulfonate 2 (614 mg, 91%). ¹H NMR (300 MHz, CDCl₃) δ 0.92 (s,3H), 1.45-1.75 (m,6H), 1.95-2.57 (m, 7H), 2.94 (m, 2H), 6.98 (s, 1H), 7.03 (d, J=8.7 Hz, 1H), 7.34 (d, J=8.7 Hz, 1H) ppm. ¹³C NMR (75 MHz, CDCl₃) δ 13.75, 21.52, 25.63, 26.04, 29.35, 31.42, 35.76, 37.68, 44.03, 47.82, 50.31, 63.06, 118.27, 121.20, 127.17, 139.26, 140.24, 147.53, 220.46 ppm.

3-diphenyliminyl-1,3,5(10)-estratrien-17-one (3) In a Schlenk tube purged with argon, Pd₂(dba)₃ (27 mg, 3% mol), S-(−)-BINAP (28 mg, 4.5% mol), Cs₂CO₃ (456 mg, 1.4 mmol) in toluene (2 mL) were added and stirring began. To the resulting solution, 2 (403 mg, 1 mmol) and benzophenone imine (201 μL, 1.2 mmol) were added and heated at 120° C. for 3 days. The dark mixture was then cooled to room temperature and diluted with Et₂O (25 mL) and filtered over Celite and then evaporated under reduced pressure. The crude solid was purified by flash chromatography using gradient elution with CH₂Cl₂ to 3% Et₂O/CH₂Cl₂ to afford 32 mg (8%) of starting material 2 and 321 mg (74%) of imine 3 as a yellow solid. ¹H NMR (300 MHz, CDCl₃) δ 0.90 (s, 3H), 1.30-1.67 (m, 6H), 1.88-2.50 (m, 7H), 2.76 (m, 2H), 6.47 (dd, J₁=8.2 Hz, J₂=1.7 Hz, 1H), 6.55 (s, 1H), 7.02 (d, J=8.2 Hz, 1H), 7.09-7.20 (m, 2H), 7.23-7.32 (m, 3H), 7.33-7.53 (m, 3H), 7.72 (d, J=7.1 Hz, 2H) ppm. ¹³C NMR (75 MHz, CDCl₃) δ 13.86, 21.54, 25.65, 26.48, 29.25, 31.55, 35.82, 38.11, 44.09, 47.95, 50.47, 118.59, 121.86, 125.25, 127.93, 128.14, 129.58, 134.90, 136.56, 139.61, 148.09, 167.93, 220.90 ppm.

3-amino-1,3,5(10)-estratrien-17-one (4) To a solution of 3 (26 mg, 0.06 mmol) in wet THF (4 mL), one drop of conc. HCl was added (the yellow colour disappears rapidly) and the solution was stirred at room temperature for 1 h. Then, the mixture was poured into CH₂Cl₂ (30 mL) and the organic phase washed with 20% aqueous NaOH (15 mL), H₂O (30 mL), and brine, dried over magnesium sulfate, filtered, and concentrated under reduced pressure. The compound was then purified by flash chromatography (hexanes-ethyl acetate/5-1) to provide 14 mg (87%) of amine 4. ¹H NMR (300 MHz, CDCl₃) δ 0.90 (s, 3H), 1.40-1.65 (m, 6H), 2.00-2.30 (m, 5H), 2.37 (m, 1H), 2.50 (dd, J₂=18.4 Hz, J₂=8.0 Hz, 1H), 2.83 (m, 2H), 3.53 (br s, 2H), 6.46 (d, J=2.4 Hz, 1H), 6.52 (dd, J₁=2.4 Hz, J₂=8.3 Hz, 1H), 7.09 (d, J=8.3 Hz, 1H) ppm. ¹³C NMR (75 MHz, CDCl₃) δ 13.84, 21.55, 25.89, 26.57, 29.47, 31.53, 35.87, 38.44, 43.93, 48.04, 50.34, 113.05, 115.32, 126.21, 130.01, 137.37, 144.16, 221.15 ppm.

3-fluoro-1,3,5(10)-estratrien-17-one (5) To neat stirred boron trifluoride etherate (642 μL, 5.07 mmol) at −15° C. under argon was added a solution of amine 4 (910 mg, 3.38 mmol) in dry CH₂Cl₂ (10 mL). After 15 min, a solution of t-butylnitrite (482 μL, 4.05 mmol) in dry CH₂Cl₂ (5 mL) was dropwise added. The dark brown solution was stirred at −15° C. for 15 min, then at 0° C. for 30 min. Pentane was added to the solution and a gummy solid precipitated. The solvent was decanted and the residue was dried under vacuum to give a crude light brown solid. The neat solid was heated under vacuum at 70-80° C. in an oil bath for 15 min to give a crude orange solid. Purification by flash chromatography (hexanes-ethyl acetate/9-1) gave fluoride 5 as a white solid (437 mg, 47%). ¹H NMR (300 MHz, CDCl₃) δ 0.92 (s, 3H), 1.37-1.70 (m, 6H), 1.93-2.44 (m, 6H), 2.52 (dd, J₁=18.8 Hz, J₂=9.0 Hz, 1H), 2.91 (m, 2H), 6.82 (m, 2H), 7.23 (m, 1H) ppm. ¹³C NMR (75 MHz, CDCl₃) δ 13.83, 21.57, 25.89, 26.31, 29.47, 31.52, 35.85, 38.09,43.98, 47.95, 50.36, 112.50 (d, J=20.9 Hz), 115.13 (d, J=19.7 Hz), 126.79 (d, J=8.4 Hz), 135.31, 138.71, 160.98 (d, J=244.3 Hz), 220.80 ppm.

3-fluoro-16,16-dimethyl-1,3,5(10)-estratrien-17-one (6) To a stirred solution of 5 (108 mg, 0.397 mmol) in dry THF (10 mL) at 0° C. was dropwise added LiHMDS (1.0 M in THF, 1.19 mL) under argon. The solution was stirred at room temperature for 30 min then cooled down to −78° C. for the addition of iodomethane (148 μL, 344 mg, 2.38 mmol). The solution was then stirred at −78° C. for 5 h and allowed to rise to room temperature overnight. The reaction was quenched with ice/water and extracted with ethyl acetate. The combined organic phase was washed with aqueous saturated NH₄Cl, 1M aqueous Na₂SO₃, H₂O, brine, dried over magnesium sulfate, filtered then rotary evaporated to give a crude solid. Purification by flash chromatography (hexanes-ethyl acetate/19-1) gave the compound 6 (82 mg, 69%) as a solid. ¹H NMR (300 MHz, CDCl₃) δ 0.94 (s, 3H), 1.08 (s, 3H), 1.21 (s, 3H), 1.42-1.63 (m, 6H), 1.86-2.00 (m, 3H), 2.28 (m, 1H), 2.40 (m, 1H), 2.89 (m, 2H), 6.78-6.87 (m, 2H), 7.22 (m, 1H) ppm. ¹³C NMR (75 MHz, CDCl₃) δ 14.42, 25.74, 25.96, 26.46, 27.29, 29.46, 32.25, 37.56, 44.15, 45.31, 47.16, 48.97, 63.31, 112.40 (d, J=20.8 Hz), 115.09 (d, J=20.9 Hz), 126.69 (d, J=7.3 Hz), 135.42, 138.65 (d, J=7.0 Hz), 160.96 (d, J=244.1 Hz), 225.03 ppm.

3fluoro-16,16-dimethyl-1,3,5(10)estratrien-17β-ol (EM-3497) To the ketone 6 (1.16 g, 3.86 mmol) in dry THF (100 mL) was added dropwise LiAlH₄ (1.0M in THF, 3.86 mL) at −78° C. The solution was stirred for 15 min then quenched with sodium sulfate decahydrate (6.22 g, 19.3 mmol) and stirred overnight. The suspension was filtered and concentrated under reduced pressure. The crude product was then purified by flash chromatography (hexanes-ethyl acetate/7-3) and triturated in cold hexanes to provide EM-3497 (1.02 g, 88%) as a white solid. IR (NaCl): 3430, 2926, 2866, 1588, 1494 cm⁻¹. ¹H NMR (300 MHz, CDCl₃) δ 0.80 (s, 3H), 1.03 (s, 3H), 1.09 (s, 3H), 1.20-1.60 (m, 8H), 1.86 (m, 1H), 1.94 (m, 1H),2.24-2.32 (m, 2H), 2.84 (m, 2H), 3.28 (d, J=7.6 Hz 1H), 6.76-6.85 (m, 2H), 7.21 (m, 1H) ppm. ¹³C NMR (75 MHz, CDCl₃) δ 11.48, 25.27, 26.14, 27.14, 29.59, 32.30, 37.78, 37.93, 38.96, 41.15, 44.00, 45.37, 46.78, 88.79, 112.26 (d, J=21.0 Hz), 115.00 (d, J=20.5 Hz), 126.71 (d, J=8.6 Hz), 135.99, 138.85 (d, J=6.9 Hz), 160.87 (d, J=243.2 Hz) ppm.

### EXAMPLE 2

Synthesis of 4-cyano-16β-ethyl-16a-methy-1,3,5(10)-estratrien-17β-ol (EM-3221)

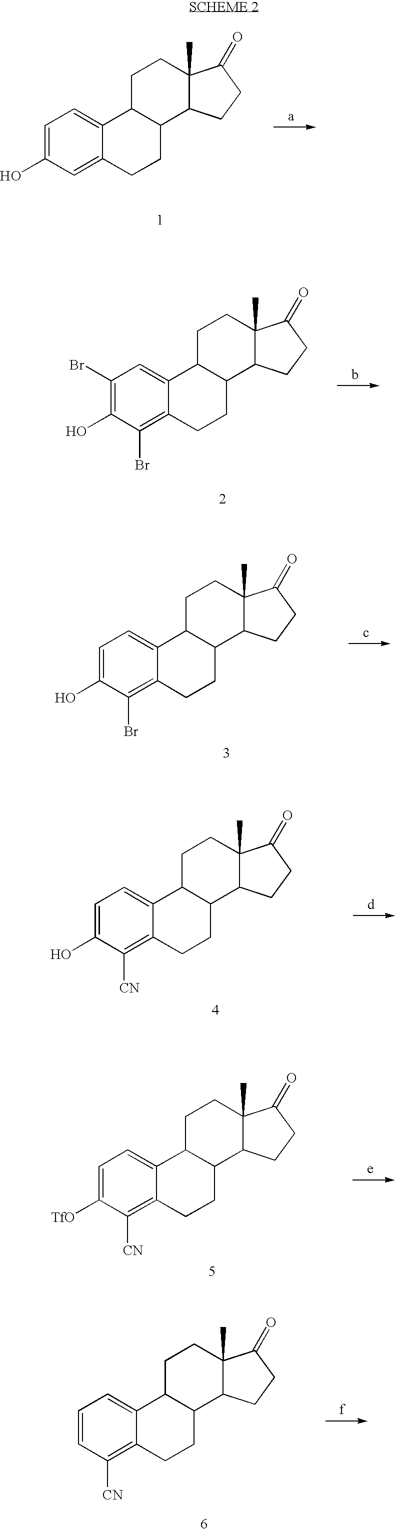

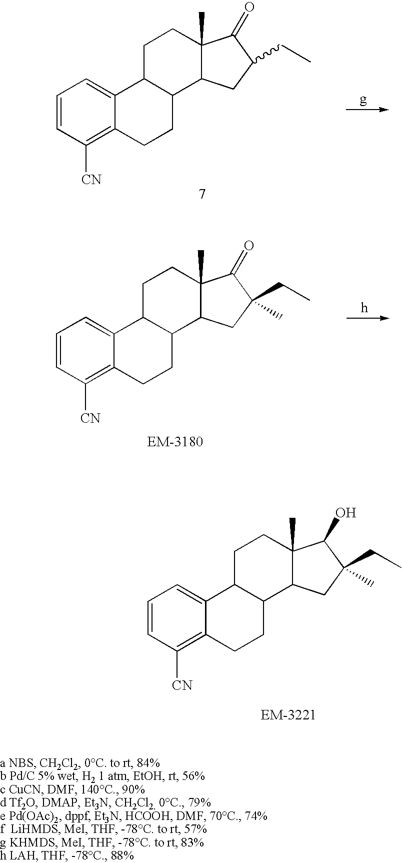

Overall yield: 10.3% (8 steps not optimized)

2,4-dibromo-3-hydroxy-1,3,5(10)-estratrien-17-one (2) Under argon atmosphere, a solution of estrone (200 g, 0.74 mol) in dry dichloromethane (3 L) was mechanically stirred at 0° C. for 30 min. N-bromosuccinimide (395 g, 2.22 mol) was slowly added and the resulting mixture was stirred at room temperature for 24 h. The reaction was quenched, with saturated aqueous NH₄Cl, washed three times with water, dried over MgSO₄, filtered, and evaporated. The crude product was dissolved in CH₂Cl₂ (125 mL) and methanol (500 mL) was added. The suspension was filtered to provide the compound 2 as a yellow solid (266 g, 84%). ¹H NMR (300 MHz, CDCl₃) δ 0.89 (s, 3H), 1.30-1.70 (m, 6H), 1.95-2.40 (m, 6H), 2.51 (dd, J₁=18.4 Hz, J₂=9.2 Hz, 1H), 2.67 (m, 1H), 2.92 (dd, J₁=17.8 Hz, J₂=6.0 Hz, 1H), 5.88 (br s, 1H), 7.39 (s, 1H) pp ¹³C NMR (75 MHz, CDCl₃) δ 13.75, 21.52, 26.06, 26.44, 30.92, 31.32, 35.83, 37.26, 43.86, 50.11, 106.44, 113.19, 128.52, 136.43, 165.01, 220.60 ppm.

3-hydroxyfbromo-1,3,5(10)-estratrien-17-one (3) A mixture of 2,4-dibromestrone 2 (166 g, 0.39 mol) and palladium on activated carbon (5 wt. %, wet) (66 g, 0.015 mol) in ethanol (2.7 L) was stirred under H₂ (g) (2 balloons) at room temperature for approximatively 90 min. The reaction was monitored by TLC. The mixture was filtered through Celite pad and washed several times with methanol. The methanol solution was thrown away (it contained only estrone). The Celite cake was transferred into an erlenmeyer with DMF (1 L). The suspension was stirred at 60° C. for 1 h, filtered on Celite, and washed with DMF. The DMF solution was poured in iced water. The suspension was filtered on Buchner funnel and dried to give the product 3 (166 g, 56%). ¹H NMR (300 MHz, CDCl₃) δ 0.90 (s, 3H), 1.40-1.85 (m, 6H), 1.90-2.45 (m, 6H), 2.51 (dd, J₁=18.3 Hz, J₂=9.2 Hz, 1H), 2.75 (m, 1H), 2.96 (dd, J₁=17.6 Hz, J₂=6.1 Hz, 1H), 5.56 (br s, 1H), 6.87 (d, J=8.5 Hz, 1H), 7.18 (d, J=8.6 Hz, 1H) ppm. ¹³C NMR (75 MHz, CDCl₃) δ 13.78, 21.52, 26.11, 26.59, 31.01, 31.45, 35.86, 37.48, 44.07, 50.17, 112.81, 125.54, 136.21, 150.20, 220.88 ppm.

3-hydroxyA4cyano1,3,5(10)-estratrien-17-one (4) A mixture of 4-bromoestrone 3 (76.1 g, 0.218 mol) and copper (I) cyanide (39.0 g, 0.436 mol) in dry DMF (1.5 L) was stirred at 140° C. for 16 h. The mixture was cooled to room temperarure and poured in a solution of FeCl₃ (320 g, 1.18 mol) in concentrated HCl (700 mL). This solution was stirred at 60° C. for 30 min, then cooled to room temperature, diluted with water, and extracted with ethyl acetate (2×2 L). The combined organic layer was washed with water and with saturated aqueous NaHCO₃ solution (until pH 8), and dried over MgSO₄. Removal of solvents gave the product 4 as a white solid (58.0 g, 90%). ¹H NMR (300 MHz, CDCl₃) δ 0.91 (s, 3H), 1.40-1.75 (m, 6H), 1.95-2.25 (m, 5H), 2.36 (m, 1H), 2.53 (dd, J₁=18.5 Hz, J₂=9.0 Hz, 1H), 2.97 (m, 1H), 3.09 (m, 1H), 5.69 (s br, 1H), 6.78 (d, J=8.7 Hz, 1H), 7.38 (d, J=8.8 Hz, 1H) ppm. ¹³C NMR (75 MHz, CDCl₃) δ 13.79, 21.50, 25.79, 28.51, 31.34, 35.82, 37.62, 43.59, 47.86, 50.08, 99.76, 113.44, 115.42, 131.46, 132.93, 140.80, 156.54, 220.77 ppm.

20 3-trifluoromethanesulfonate-4-cyano-1,3,5(10)-estratrien-17-one (5) Under argon atmosphere, a solution of compound 4 (8.0 g, 26 mmol), triethylamine (7.1 mL, 52 mmol) and 4dimethylaminopyridine (0.35 g, 2.6 mmol) in dichloromethane (700 mL) was cooled at 0° C., treated with trifluoromethanesulfonic anhydride (5.2 mL, 31 mmol), and stirred for 45 min. The reaction mixture was quenched with water and extracted with dichloromethane. The organic phase was washed with 2% hydrochloric acid, saturated aqueous NaHCO₃ solution and water, dried over MgSO₄, filtered, and evaporated. The crude product was purified by flash chromatography (hexanes-ethyl acetate-CHCl₃/8-1-1 to hexanes-ethyl acetate-CHCl₃/7-2-1) to provide sulfonate 5 (8.7 g, 79%). ¹H NMR (300 MHz, CDCl₃) δ 0.92 (s, 3H), 1.45-1.80 (m, 6H), 1.98-2.30 (m, 4H), 2.35-2.50 (m, 2H), 2.53 (dd, J₁=18.3 Hz, J₂=9.1 Hz, 1H), 3.06 (m, 1H), 3.22 (dd, J₁=18.2 Hz, J₂=5.5 Hz, 1H), 7.25 (d, J=8.3 Hz, 1H), 7.60 (d, J=8.9 Hz, 1H) ppm. ¹³C NMR (75 MHz, CDCl₃) δ 13.70, 21.48, 25.44, 25.58, 28.59, 29.68, 31.24, 35.70, 37.02, 43.87, 47.64, 50.09, 107.08, 112.74, 119.30, 131.31, 141.45, 143.50, 148.04, 219.92 ppm.

4-cyano-1,3,5(10)-estratrien-17-one (6) A round-bottom flask charged with triflate 5 (8.4 g 20 mmol), Et₃N (8.2 mL, 59 mmol), 1,1′-bis(diphenylphosphino)ferrocene (218 mg 0.39 mmol), palladium acetate (441 mg 0.20 mmol) suspended in dry DMF (130 mL), and formic acid (1.5 mL, 39 mmol) was heated, under an argon atmosphere, at 70 ° C. overnight. The reaction mixture was quenched with water and extracted with ethyl acetate. The organic phase was washed with water and brine, dried over MgSO₄, filtered, and evaporated. The crude product was purified by flash chromatography (hexanes-ethyl acetate-CHCl₃/8-1-1 to hexanes-ethyl acetate-CHCl₃/5-5-1) to provide nitrile 6 (4.1 g, 74%). ¹H NMR (300 MHz, CDCl₃) δ 0.92 (s, 3H), 1.40-1.75 (m, 6H), 1.90-2.25 (m, 4H), 2.25-2.50 (m, 2H), 2.53 (dd, J₁=18.2 Hz, J₂=9.3 Hz, 1H), 3.02 (m, 1H), 3.20 (dd, J₁=17.8 Hz, J₂=5.9 Hz, 1H), 7.24 (m, 1H), 7.51 (m, 2H) ppm. ¹³C NMR (75 MHz, CDCl₃) δ 13.78, 21.52, 25.62, 25.78, 28.18, 31.38, 35.80, 37.36, 44.17, 47.78, 50.26, 112.57, 118.13, 126.19, 129.93, 130.39, 140.28, 141.25, 220.87 ppm.

4-cyano16-ethyl-1,3,5(10)-estratrien-17-one (7) To a stirred solution of 6 (500 mg, 1.79 mmol) in dry THF (18 mL) at 0° C. was dropwise added LiHMDS (1.0 M in THF, 1.88 mL) under an argon atmosphere. The solution was stirred at room temperature for 30 min then cooled down to −78° C. for the addition of iodoethane (157 μL, 307 mg, 1.97 mmol). The solution was then stirred at room temperature for 7 h. The reaction was quenched with ice/water and extracted with ethyl acetate. The combined organic phase was washed with aqueous saturated NH₄Cl, 1M aqueous Na₂SO₃, H₂O and brine, dried over MgSO₄, filtered, then rotary evaporated to give a crude solid. Purification by flash chromatography (hexanes-ethyl acetate/19-1 to hexanes-ethyl acetate/4-1) gave the compound 7 (315 mg, 57%) as a solid. The ratio α/β isomer is 5/1; the pure α-ethyl isomer is described. ¹H NMR (300 MHz, CDCl₃) δ 0.95-1.00 (m, 6H), 1.25-1.70 (m, 6H), 1.80-2.00 (m, 4H), 2.13 (m, 1H), 2.30 (m, 1H), 2.40-2.50 (m, 2H), 3.01 (m, 1H), 3.20 (dd, J₁=18.0 Hz, J₂=5.9 Hz, 1H), 7.23 (m, 1H), 7.50 (m, 2H) ppm. ¹³C NMR (75 MHz, CDCl₃) δ 12.53, 14.55, 24.01, 25.53, 25.75, 26.81, 28.21, 31.45, 37.32, 44.20, 46.41, 48.31, 48.39, 112.53, 118.17, 126.16, 129.92, 130.33, 140.28, 141.34, 221.78 ppm.

4-cyano-16β-ethyl-16α-methyl-1,3,5(10)-estratrien-17one (EM-3180) To a stirred solution of 7 (220 mg, 0.716 mmol) in dry THF (14 mL) at 0° C. was dropwise added KHMDS (0.5 M in toluene, 2.14 mL) under an argon atmosphere. The solution was stirred at room temperature for 30 min then cooled down to −78° C. for the addition of iodomethane (134 μL, 305 mg, 2.15 mmol). The solution was then stirred at −78° C. for 30 min and allowed to rise to room temperature for 2 h. The reaction was quenched with ice/water and extracted with ethyl acetate. The combined organic phase was washed with aqueous saturated NH₄Cl, 1M aqueous Na₂, SO₃, H₂O and brine, dried over MgSO₄, filtered, then rotary evaporated to give a crude solid. Purification by flash chromatography (hexanes-ethyl acetate/19-1 to hexanes-ethyl acetate/9-1) gave the compound EM-3180 as a solid (199 mg, 83%). ¹H NMR (300 MHz, CDCl₃) δ 0.92 (m, 6H), 1.06 (s, 3H), 1.40-1.70 (m, 8H), 1.80 (m, 1H), 2.01 (m, 1H), 2.11 (m, 1H), 2.30-2.50 (m, 2H), 3.02 (m, 1H), 3.20 (dd, J₁=17.6 Hz, J₂=5.1 Hz, 1H), 7.22 (m, 1H), 7.50 (m, 2 ppm. ¹³C NMR (75 MHz, CDCl₃) δ 8.97, 14.20, 22.53, 25.55, 26.02, 28.23, 32.36, 34.85, 36.88, 44.43, 47.10, 48.73, 49.63, 112.58, 118.18, 126.18, 129.85, 130.35, 140.33,141.45,224.69 ppm.

4 cyano-16β-ethyl-16α-methyl-1,3,5(10)-estratrien-17β-l (EM-3221) To the ketone EM-3180 (60 mg, 0.187 mmol) in dry THF (4 mL) was added dropwise LiAlH₄ (1.0 M in THF, 206 μL) at −78° C. The solution was stirred for 15 min, then quenched with sodium sulfate decahydrate (303 mg, 0.94 mmol), and stirred overnight. The suspension was filtered and concentrated under reduced pressure. The crude product was then purified by flash chromatography (hexanes-ethyl acetate/19-1 tohexanes-ethyl acetate/5-1) to give the compound EM-3221 (53 mg, 88%). ¹H NMR (300 MHz, CDCl₃) δ 0.79 (s, 3H), 0.92 (t, J=7.4 Hz, 3H), 1.05 (s, 3H), 1.25 (m, 1H), 1.30-1.50 (m, 8H), 1.97 (m, 2H), 2.31 (m, 2H), 2.97 (m, 1H), 3.15 (m, 1H), 3.33 (d, J=7.8 Hz, 1H), 7.20 (m, 1iH), 7.50 (m, 2H) ppm. ¹³C NMR (75 MHz, CDCl₃) δ 8.77, 11.66, 25.90, 26.66, 27.81, 28.34, 29.71, 37.11, 37.89, 38.27, 42.30, 44.23, 45.19, 46.81, 90.95, 112.45, 118.27,126.00,129.92, 130.14,140.53, 141.96 ppm.

### PHARMACEUTICAL COMPOSITION EXAMPLES

Set forth below, by way of example and not of limitation, are several pharmaceutical compositions utilizing a preferred active compound EM-3180. Other compounds of the invention or combination thereof, may be used in place of (or in addition to) EM-3180. The concentration of active ingredient may be varied over a wide range as discussed herein. The amounts and types of other ingredients that may be included are well known in the art.

### EXAMPLE A

Composition suitable for use as topical lotion

| | Weight % |
|---|---|
| Ingredient | (by weight of total composition) |
| EM-3180 | 1.0 |
| Ethanol | 70.0 |
| Propylene glycol | 29.0 |

### EXAMPLE B

Composition suitable for use as topical gel

| | Weight % |
|---|---|
| Ingredient | (by weight of total composition) |
| EM-3180 | 1.0 |
| Kucel | 1.5 |
| Ethanol | 70.0 |
| Propylene glycol | 27.5 |

### EXAMPLE C

Composition suitable for use as topical gel

| | Weight % |
|---|---|
| Ingredient | (by weight of total composition) |
| EM-3180 | 1.0 |
| Finasteride | 1.0 |
| Ethanol | 69.0 |
| Propylene glycol | 29.0 |

### EXAMPLE D

Composition suitable for use as topical gel

| | Weight % |
|---|---|
| Ingredient | (by weight of total composition) |
| EM-3180 | 1.0 |
| Finasteride | 1.0 |
| Kucel | 1.5 |
| Ethanol | 69.0 |
| Propylene glycol | 27.5 |

### EXAMPLE E

Composition suitable for use as topical gel

| | Weight % |
|---|---|
| Ingredient | (by weight of total composition) |
| EM-3180 | 1.0 |
| EM-1404 | 2.0 |
| Ethanol | 68.0 |
| Propylene glycol | 29.0 |

### EXAMPLE F

Composition suitable for use as topical gel

| | Weight % |
|---|---|
| Ingredient | (by weight of total composition) |
| EM-3180 | 1.0 |
| EM-1404 | 2.0 |
| Kucel | 1.5 |
| Ethanol | 68.0 |
| Propylene glycol | 27.5 |

### EXAMPLE G

Composition suitable for use as topical gel

| | Weight % |
|---|---|
| Ingredient | (by weight of total composition) |
| EM-3180 | 1.0 |
| BM-1791 | 2.0 |
| Ethanol | 68.0 |
| Propylene glycol | 29.0 |

### EXAMPLE H

Composition suitable for use as topical gel

| | Weight % |
|---|---|
| Ingredient | (by weight of total composition) |
| EM-3180 | 1.0 |
| EM-1791 | 2.0 |
| Kucel | 1.5 |
| Ethanol | 68.0 |
| Propylene glycol | 27.5 |

Other antiandrogens may be substituted for or added to EM-3180 in the above formulations.

The invention has been described in terms of preferred embodiments and examples, but is not limited thereby. Those of skill in the art will readily recognize the broader applicability and scope of the invention which is limited only by the patent claims that issue from this application or any patent application claiming priority (directly or indirectly) hereto.

What is claimed:

1. A compound having the molecular structure: 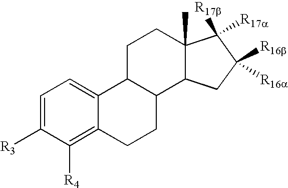
wherein R₃ is selected from the group consisting of hydrogen, fluoro, chloro, bromo, iodo, and a moiety —C≡CR′ (R′ being hydrogen or C1-C6 lower alkyl);
wherein R₄ is selected from the group consisting of hydrogen, fluoro, chloro, bromo, iodo, and cyanide;
wherein R_{17α} is selected from the group consisting of hydrogen, C1-C6 lower alkyl, C2-C6 lower alkenyl, and C2-C6 lower alkynyl, or R_{17α} and R_{17β} together are oxygen forming a keto group;
wherein R_{17β} is selected from the group consisting of hydroxyl and a group transformed on the skin into hydroxyl, or R_{17α} and R_{17β} together are oxygen forming a keto group;
wherein R_{16α} is selected from the group consisting of hydrogen, C1-C6 lower alkyl C2-C6 lower alkenyl, and C2-C6 lower alkynyl;
whereinm R_{16β} is selected from the group consisting of hydrogen, C1-C6 lower alkyl, C2-C6 lower alkenyl, and C2-C6 lower alkynyl;
wherein at least one of R₃ or R₄is not an hydrogen.

2. The compound selected from the group consisting of: 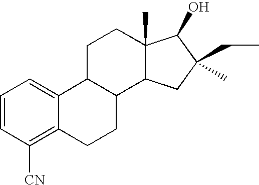
4-cyano-16α-methyl-16β-ethyl-1,3,5(10)estratrien-17β-ol and 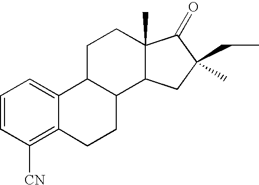
4-cyano-16α-methyl-16β-ethyl-1,3,5(10)-estratrien-17-one

3. A pharmaceutical composition comprising a pharmaceutical acceptable diluent or carrier and a therapeutically acceptable amount of an antiandrogen having the molecular structure: 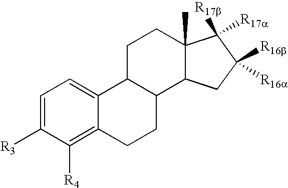
wherein R₃ is selected from the group consisting of hydrogen, fluoro, chloro, bromo, iodo, and a moiety —C≡CR′ (R′ being hydrogen or C1-C6 lower alkyl);
wherein R₄ is selected from the group consisting of hydrogen, fluoro, chloro, bromo, iodo, and cyanide;
wherein R_{17α} is selected from the group consisting of hydrogen, C1-6 lower alkyl, C2-C6 lower alkenyl, and C2-C6 lower alkynyl, or R_{17α} and R_{17β} together are oxygen forming a keto group;
wherein R_{17β} is selected from the group consisting of hydroxyl and a group transformed on the skin into hydroxyl, or R_{17α} and R_{7β} together are oxygen forming a keto group;
wherein R_{16α} is selected from the group consisting of hydrogen, C1-C6 lower alkyl, C2-C6 lower alkenyl, and C2-C6 lower alkynyl;
wherein R_{16β} is selected from the group consisting of hydrogen, C1-6 lower alkyl, C2-C6 lower alkenyl, and C2-C6 lower alkynyl;
wherein at least one of R₃, or R₄ is not an hydrogen.

4. A pharmaceutical composition comprising a pharmaceutical acceptable diluent or carrier and a therapeutically acceptable amount of an antiandrogen selected from the group consisting of: 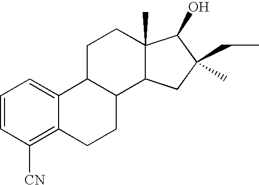
4-cyano-16α-methyl-16β-ethyl-1,3,5(10)-estratrien-17β-ol and 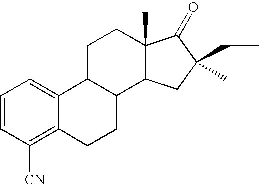
4-cyano-16α-methyl-16β-ethyl-1,3,5(10)-estratrien-17-one

5. A method of treating or reducing the risk of developing, acne, seborrhea, hirsutism or androgenic alopecia, comprising administering to a patient in need of such treatment or reduction, a therapeutically effective amount of the compound of claim 1

6. The method of claim 5, further comprising administering to said patient a therapeutically effective amount of an inhibitor of type 5 17β-hydroxysteroid dehydrogenase

7. The method of claim 5, further comprising administering to said patient a therapeutically effective amount of a 5α-reductase inhibitor.

8. The method of claim 5, further comprising administering to said patient a therapeutically effective amount of an inhibitor of Prostate Short-Chain Dehydrogenase/Reductase 1 (PSDR1).

9. The method of claim 6, further comprising administering to said patient a therapeutically effective amount of an inhibitor of Prostate Short-Chain Dehydrogenase/Reductase 1 (PSDR1).

10. The method of claim 7, further comprising administering to said patient a therapeutically effective amount of an inhibitor of Prostate Short-Chain Dehydrogenase/Reductase 1 (PSDR1).

11. The method of claim 5, further comprising administering to said patient a therapeutically effective amount of a 5α-reductase inhibitor and an inhibitor of type 5 17□-hydroxysteroid dehydrogenase.

12. The method of claim 11, further comprising admininistering to said patient a therapeutically effective amount of an inhibitor of Prostate Short-Chain Dehydrogenase/Reductase 1 (PSDR1).
